(19) United States
(12) Patent Application Publication
(10) Pub. No.: US 2014/0074127 A1
(43) Pub. Date: Mar. 13, 2014

(54) SOFT TISSUE CONNECTOR

(71) Applicant: Zimmer, Inc., Warsaw, IN (US)

(72) Inventors: Michael Giordano, South Bend, IN (US); Miranda Shaw, Warsaw, IN (US)

(73) Assignee: Zimmer, Inc., Warsaw, IN (US)

(21) Appl. No.: 14/016,377

(22) Filed: Sep. 3, 2013

Related U.S. Application Data
(60) Provisional application No. 61/698,044, filed on Sep. 7, 2012.

(51) Int. Cl. A61B 17/08

(52) U.S. Cl. CPC A61B 17/08
USPC 606/151

(57) ABSTRACT

Various embodiments of the present disclosure include a soft tissue connector comprising a substrate having first and second surfaces. The first surface can include at least one bone penetrating member for securing the substrate to a bone. The second surface can include a topography configured to allow releasable attachment of soft tissue to the substrate. Methods and kits for facilitating connection of soft tissue to bone are also provided.

### CLAIM OF PRIORITY

This application claims the benefit of U.S. Provisional Patent Application Ser. No. 61/698,044, filed on Sep. 7, 2012, which is hereby incorporated by reference herein in its entirety.

### BACKGROUND

The successful reattachment of soft tissue to bone can be a significant concern, especially in the sports medicine industry.

The majority of soft tissue repairs involve suture anchors or tacks. In such methods, an anchor or tack is readied for insertion into bone and a suture is passed through tissue and the anchor or tack. While holding tension on the suture, joint stability is evaluated and the anchor or tack is deployed into the bone, finalizing the repair.

In one approach, suture deployment tools relate the tension on the tissue to the depth at which the anchor or tack is inserted into bone. Other methods provide more user-friendly methods of tensioning, but do not allow tension to be reduced in the case of over tightening.

### SUMMARY

The present inventors have recognized, among other things, that a problem to be solved can include adjustable tissue tensioning. Existing apparatus for, and methods of, soft tissue repair do not allow for easy tissue tensioning and, if needed or desired, adjustment of the tensioning. Only one known anchor or tack currently on the market (i.e., Smith & Nephew's FOOTPRINT™) allows a surgeon to decrease an initial tension, although, even through use of this anchor, a tensioning tool must remain connected to the anchor during the process. Once the tensioning tool is removed, the anchor and suture tension are set and no further adjustment can be made. The present subject matter can help provide a solution to this problem, such as by allowing the surgeon increased flexibility when tensioning and, if needed, adjusting the tension of a tissue repair.

The present disclosure relates generally to soft tissue connectors, kits, and methods that allow the adjustment of tension between soft tissue and bone. To better illustrate the soft tissue connectors, kits, and methods disclosed herein, a non-limiting list of examples is provided here:

In Example 1, a soft tissue connector comprises a substrate having first and second surfaces. The first surface can include at least one bone penetrating member for securing the substrate to a bone. The second surface can include topography configured to allow releasable attachment of soft tissue to the substrate.

In Example 2, the connector of Example 1 is optionally configured such that the first surface includes at least two bone penetrating members.

In Example 3, the connector of Example 2 is optionally configured such that the first bone penetrating member extends from a first end of the first surface, and a second bone penetrating member extends from an opposing second end of the first surface.

In Example 4, the connector of Example 3 is optionally configured such that each of the first and second bone penetrating members includes a bend of the substrate. The bends can terminate in one or more teeth.

In Example 5, the connector of any one or any combination of Examples 1-4 is optionally configured such that at least a portion of the substrate is flexible for fitting about a non-linear contour of the bone.

In Example 6, the connector of any one or any combination of Examples 1-5 is optionally configured such that the substrate includes a planar sheet of material.

In Example 7, the connector of any one or any combination of Examples 1-5 is optionally configured such that the substrate includes a pre-formed curvature.

In Example 8, the connector of any one or any combination of Examples 1-7 is optionally configured such that the topography includes a plurality of angle projections extending at a non-perpendicular orientation relative to the second surface.

In Example 9, the connector of any one or any combination of Examples 1-7 is optionally configured such that the topography includes one or more teeth.

In Example 10, the connector of any one or any combination of Examples 1-7 is optionally configured such that the topography includes a hook and/or loop fastener material.

In Example 11, the connector of any one or any combination of Examples 1-7 is optionally configured such that the topography includes a cellular structure of the second surface.

In Example 12, the connector of any one or any combination of Examples 1-11 is optionally configured such that the substrate includes a bioresorbable material.

In Example 13, a kit comprises a soft tissue connector, a fixation tool, and a set of instructions. The soft tissue connector can include a substrate having opposing first and second surfaces. The first surface can include at least one bone penetrating member for securing the substrate to a bone. The second surface can include topography configured to allow releasable attachment of soft tissue to the substrate. The fixation tool can be configured to secure the at least one bone penetrating member to the bone. The set of instructions can include instructions for using one or both of the soft tissue connector or the fixation tool.

In Example 14, a method for facilitating connection of soft tissue to bone comprises obtaining a soft tissue connector including a substrate having first and second surfaces. The first surface can include at least one bone penetrating member for securing the substrate to a bone. The second surface can include topography to allow attachment of soft tissue to the substrate. The method can further include securing the at least one bone penetrating member to the bone and attaching soft tissue to the topography. The attachment of the soft tissue to the topography can effectuate a first tissue tensioning.

In Example 15, the method of Example 14 optionally further comprises testing one or more of a stability or a range of motion of a joint, associated with the bone, when the soft tissue has the first tissue tensioning.

In Example 16, the method of Example 15 optionally further comprises adjusting the tension of the soft tissue if the testing result is not satisfactory. Adjusting the tension of the soft tissue can include releasing the soft tissue from the topography and re-attaching the soft tissue to the topography to effectuate a second tissue tensioning, which is different than the first tissue tensioning.

In Example 17, the method of Example 16 optionally further comprises testing one or more of the stability or the range of motion of the joint when the soft tissue has the second tissue tensioning.

In Example 18, the method of any one or any combination of Examples 14-17 is optionally configured such that attaching the soft tissue to the topography includes releasably attaching the soft tissue to the soft tissue connector without causing tissue damage.

In Example 19, the method of any one or any combination of Examples 14-18 optionally further comprises driving one or more tacks through the soft tissue and into the bone to secure a position of the soft tissue.

In Example 20, the method of any one or any combination of Examples 14-19 optionally further comprises positioning a second soft tissue connector on an outward facing surface of the soft tissue and securing the second soft tissue connector to the first soft tissue connector or to the bone.

In Example 21, a soft tissue connector comprises a substrate having first and second surfaces. At least one of the first and second surfaces includes topography configured to allow releasable attachment of soft tissue to the substrate.

In Example 22, the soft tissue connector, kit, or method of any one or any combination of Examples 1-21 is optionally configured such that all elements or options recited are available to use or select from.

These and other examples and features of the present soft tissue connectors, kits, and methods will be set forth in part in the following Detailed Description. This Summary is intended to provide non-limiting examples of the present subject matter—it is not intended to provide an exclusive or exhaustive explanation. The Detailed Description below is included to provide further information about the present soft tissue connectors, kits, and methods.

### BRIEF DESCRIPTION OF DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 is a side view of a soft tissue connector, according to example embodiments.

FIG. 2 is a pictorial view of a soft tissue connector, according to example embodiments.

FIGS. 3A-3F show aspects of a method for facilitating connection of soft tissue to bone, according to example embodiments.

FIGS. 4A-4B show aspects of a method for facilitating connection of soft tissue to bone, according to example embodiments.

FIG. 5A-5B show aspects of a method for facilitating connection of soft tissue to bone, according to example embodiments.

### DETAILED DESCRIPTION

Reference is first made to FIG. 1 of the accompanying drawings. In an example embodiment, a soft tissue connector 10 can comprise a substrate 12 having opposing first and second surfaces 14 and 16. The first surface 14 can include at least one bone penetrating member for securing the substrate 12 to a bone. In the illustrated example, two bone penetrating members 18A and 18B are provided, but more or less are possible. The second surface 16 can include a surface topography, shown generally at 20, configured to allow releasable attachment of soft tissue to the substrate 12. A bone and soft tissue are not shown in FIG. 1, but are referred to in later figures and described in further detail below.

In various embodiments, at least a portion of the substrate 12 can be flexible for fitting about a non-linear contour of a bone. In some embodiments, the substrate 12 can include a planar sheet of material. In some embodiments, the substrate 12 can include a pre-formed curvature.

Each bone penetrating member 18A and 18B can have a wedge or other bone penetrable shape that defines a sharp point to facilitate being driven into bone. In an example embodiment, a first bone penetrating member 18A can extend from the first surface 14 at or towards a first end 22 of the first surface, and a second bone penetrating member 18B can extend from the first surface 14 at or towards an opposing second end 24 of the first surface. The illustrated bone penetrating members 18A and 18B are, in this example, formed integrally with the substrate, but in various embodiments, the members can be separate elements that can be driven into bone to secure the substrate 12 to a bone.

One or more apertures can be provided in the substrate 12 to allow one or more separate bone penetrating members to pass through the substrate apertures and secure the substrate to a bone. The bone penetrating members can selectively be secured in one or more desired positions to suit the requirements of a given soft tissue repair or surgical procedure. Examples of bone penetrating members include pins and tacks. Other configurations of bone penetrating members are possible, and any number of bone penetrating members can be provided as deemed appropriate by a caregiver.

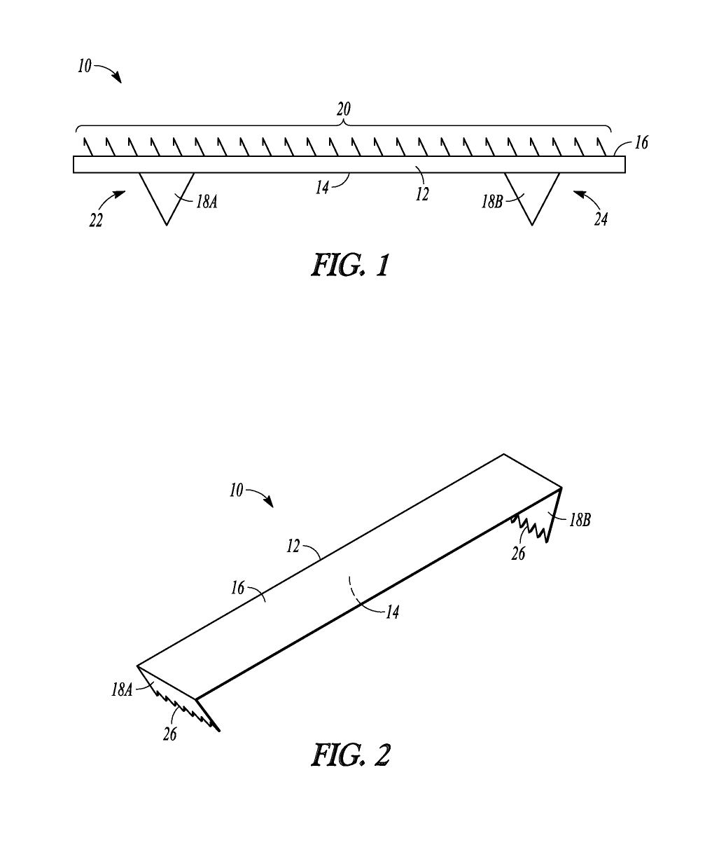

Reference is now made to FIG. 2 of the accompanying drawings, which illustrates another embodiment of a soft tissue connector 10. In the illustrated embodiment, each of the first and second bone penetrating members 18A and 18B includes a bent portion of the substrate. The bent portions can be rigidly formed, or can be deformable by a surgeon, for example, out of plane relative to the substrate's 12 main body to define bone penetrating members of different lengths and/or orientations. The bends defining the bone penetrating members 18A and 18B can terminate in one or more teeth 26.

The topography of the second surface 16 of the connector 10 can be provided in various forms. For example, the second surface 16 of the connector 10 can be textured in such a way to allow releasable or temporary attachment of soft tissue to the substrate 12. The second surface 16 of the substrate 12 can be treated or shaped to define a friction-increasing or tenacious surface. In some embodiments, the second surface 16 can be defined by a layer of special-purpose coating or other material.

In various embodiments, the topography can include a plurality of angled projections extending at a non-perpendicular orientation relative to the second surface 16 of the connector 10. The topography can include one or more teeth or various types of hook and/or loop fastener materials. In some embodiments, the topography can include a cellular or porous structure of the second surface 16 or substrate 12. Cellular or porous materials can include, for example, a material known by the trade name TRABECULAR METAL®, available from Zimmer, Inc., of Warsaw, IN, USA and described in U.S. Pat. No. 5,282,861 to Kaplan, entitled “Open Cell Tantalum Structures for Cancellous Bone Implants and Cell and Tissue Receptors,” which is incorporated herein by reference.

In some embodiments, the substrate 12 can include a bioresorbable or biodegradable material. Using a fast absorbing bioresorbable material for the substrate, for example, can facilitate full bone/tissue contact once the material has been absorbed by the body. This bone/tissue contact has, in some instances, been shown to be an important factor in proper healing.

In some embodiments, a kit of surgical tools can be provided. A kit can comprise a soft tissue connector 10 of any example embodiment described herein, a fixation tool configured to secure at least one bone penetrating member of the soft tissue connector 10 to bone, and instructions for using one or both of the soft tissue connector or the fixation tool. The fixation tool can be a multi-shot or single-shot bone pin gun, for example.

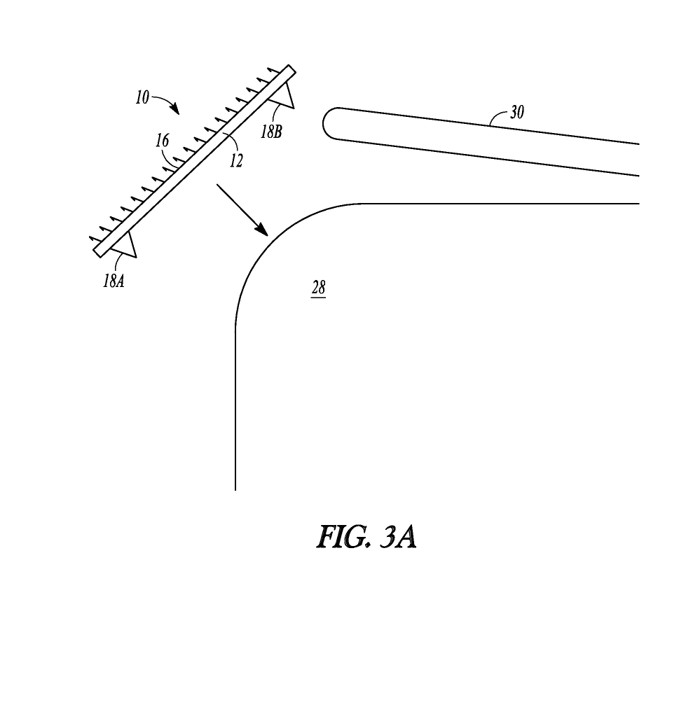

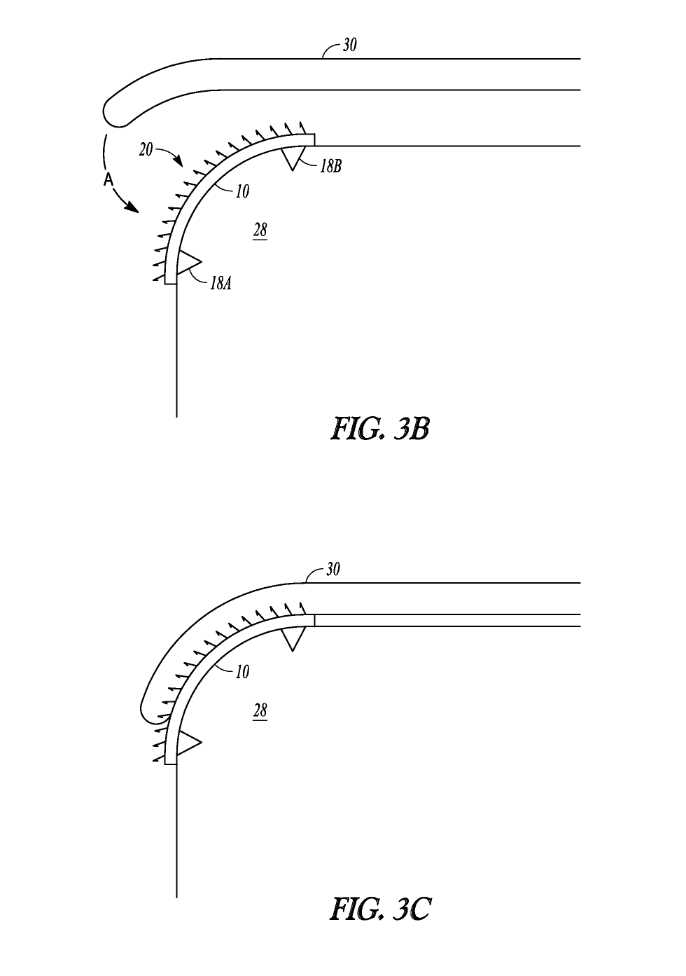

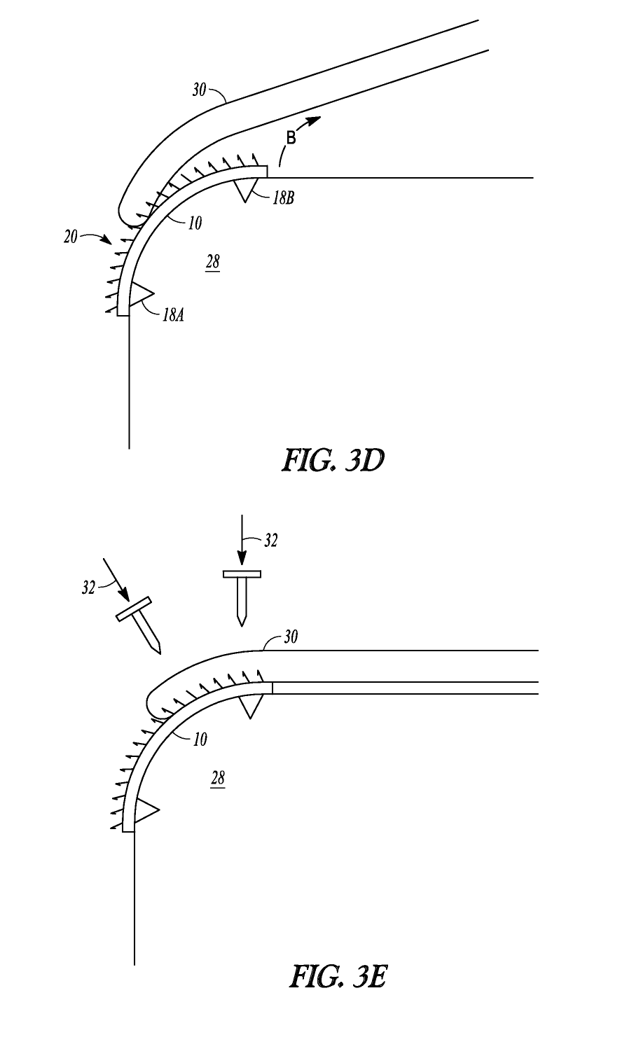

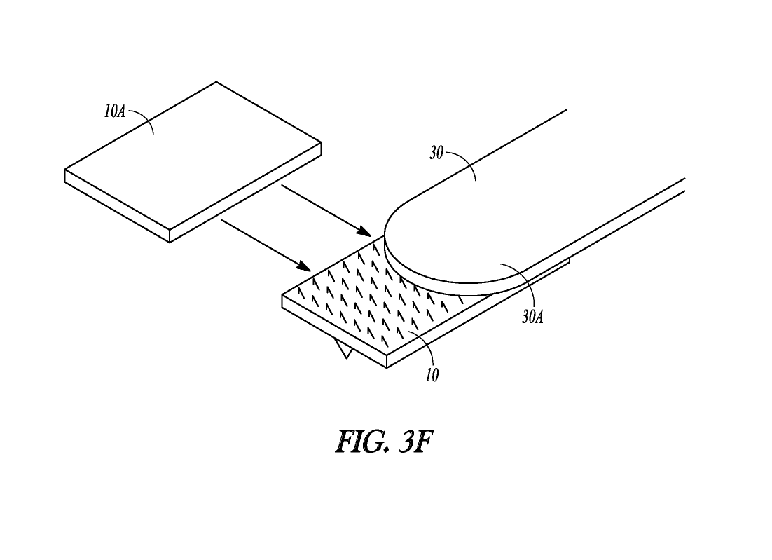

Some embodiments of the present inventive subject matter include methods for facilitating connection of soft tissue to bone. One such method embodiment is now described with reference to FIGS. 3A-3F. In this example embodiment, the method can comprise obtaining a soft tissue connector 10, for example as shown in FIG. 3A. The connector 10 can include a substrate 12 having first and second surfaces 14 and 16. At least one (separate or integral) bone penetrating member can be provided or obtained, for example the illustrated members 18A and 18B, for securing the substrate 12 to a bone 28. The second surface 16 of the connector 10 can include a topography configured to allow releasable attachment of soft tissue 30 to the substrate 12.

With reference to FIG. 3B, the method can include deforming the planar substrate 12 to conform generally to contours of the bone 28 and securing the at least one bone penetrating member (in this example, both members 18A and 18B) to the bone 28. This process can secure the connector 10 in place at a desired location on the bone 28.

With continuing reference to FIG. 3B, the method can include attaching soft tissue 30 to the topography 20 on the second surface 16 of the substrate 12 in a first position, thereby effectuating a first tissue tensioning between the soft tissue 30 and bone 28.

The soft tissue 30 can be drawn over the connector 10 to the first position in the direction of arrow A, for example, and then lowered onto the connector 10 to engage the soft tissue 30 with the topography 20. A selected first position and associated first tissue tensioning is shown, for example, in FIG. 3C. In this first position, a relatively longer portion of the soft tissue 30 is secured to the connector than a second tissue positioning illustrated and described in association with FIGS. 3D and 3E. Once the soft tissue 30 has been secured in the first position, the method can further comprise testing one or more of a stability or a range of motion of a joint, associated with the bone 28, when the soft tissue 30 has the first tissue tensioning.

With reference to FIG. 3D, the method can still further comprise, if the testing result is not satisfactory to the caregiver, adjusting the tension of the soft tissue by releasing the soft tissue from the topography 20 and re-attaching the soft tissue to the topography 20 in a second position on the connector 10, thereby effectuating a second tissue tensioning. For example, if the first tissue tensioning is too tight, the soft tissue 30 can be released from the topography 20 and moved in the direction of arrow B to a selected second position in which the tensioning is reduced. It can be seen that in this second position, a relatively shorter portion of the soft tissue 30 is secured to the connector 10 when compared to the first tissue positioning illustrated and described in association with FIGS. 3B and 3C. The method can further comprise testing one or more of the stability or the range of motion of the joint when the soft tissue has the second tissue tensioning. One or more further reattachment and testing steps can be performed until a desired location of the soft tissue is identified. In varying method embodiments, attaching the soft tissue 30 to the topography 20 can include releasably attaching the soft tissue 30 to the connector 10 without causing significant tissue damage.

As shown in FIG. 3E, the method can further comprise driving one or more tacks 32 (or the like) through the soft tissue 30 and into the bone 28 to secure a position of the soft tissue 30.

With reference to FIG. 3F, the method can further comprise positioning a second soft tissue connector 10A on an outward facing surface 30A of the soft tissue 30 and securing the second soft tissue connector 10A to the first soft tissue connector 10 or to the bone 28.

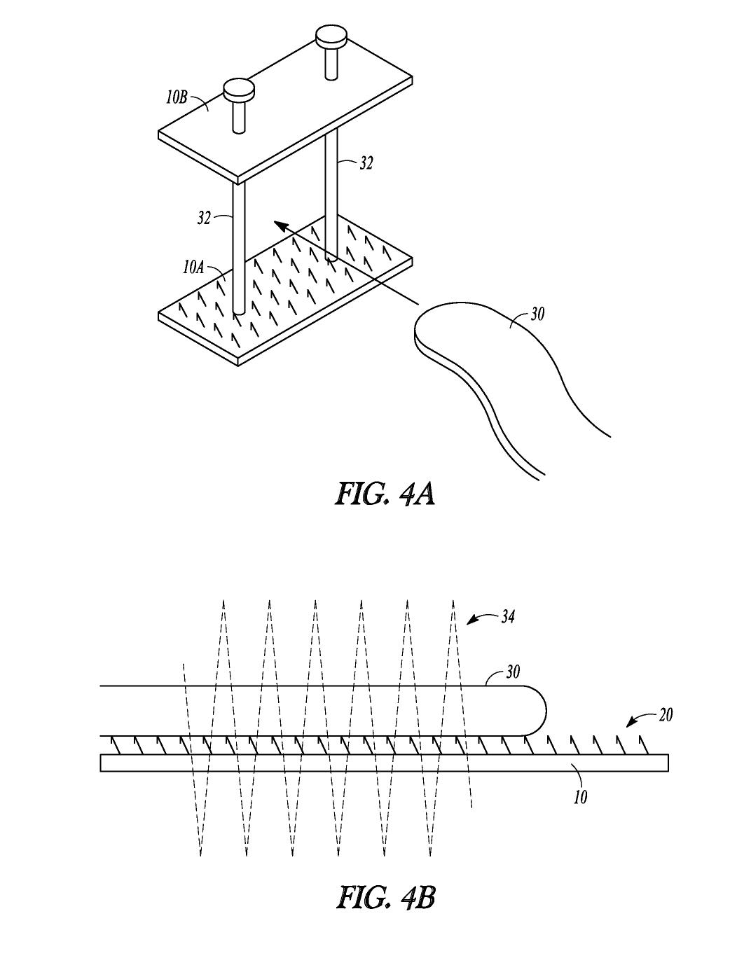

With reference to FIG. 4A, further or alternate aspects of the method can include compressing soft tissue between two connectors 10A and 10B and attaching at least one of the connectors to a bone. Compression bolts 32 can be used to compress the soft tissue 30 and/or secure one or more of the connectors 10A and 10B to a bone.

With reference to FIG. 4B, a portion of soft tissue 30 can be stitched to a connector 10. The stitching is shown generally, by way of example, at 34. The substrate material of the soft tissue connector 10 can allow stitching 34 to pass through it. The engagement of topography 20 and the tissue 30 can increase the strength of the attachment between the tissue 30 and the connector 10.

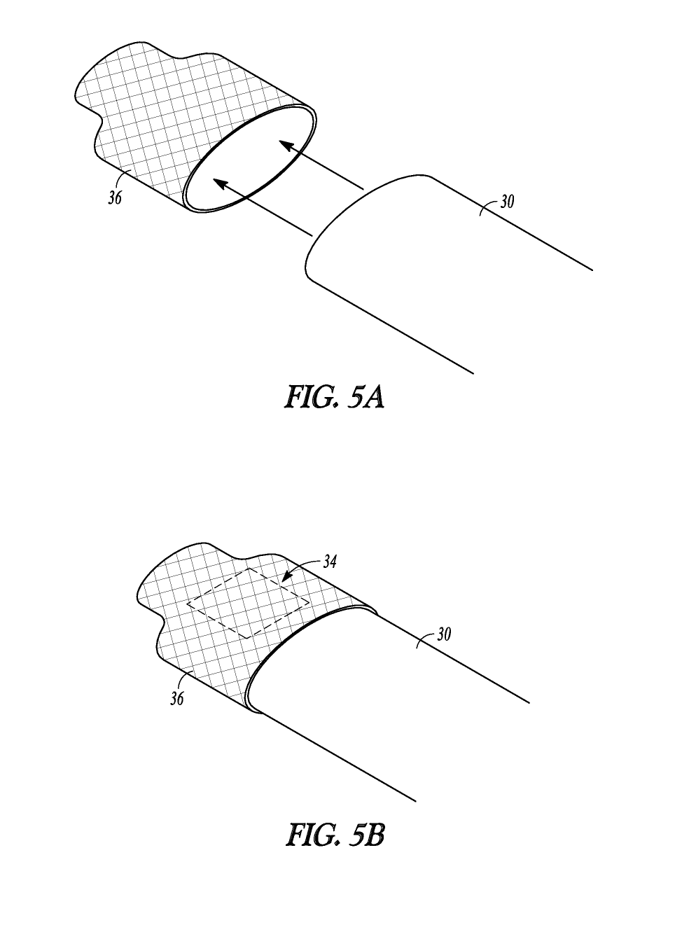

Further or alternate aspects of the method are illustrated in FIGS. 5A and 5B. In these example embodiments, an extension member can be secured to an end portion of soft tissue 30. The extension member can be in the form of a sock 36, and the sock can be secured to the tissue by stitching 34, for example. The soft tissue 30 can be joined indirectly to bone 28 using the extension member and any one of the example embodiments of a connector 10 as illustrated or described herein.

### NON-LIMITING EMBODIMENTS

While the invention has been described with reference to specific embodiments, it will be understood by those skilled in the art that various changes can be made and equivalents can be substituted for the elements thereof without departing from the true spirit and scope of the invention. In addition, modifications can be made without departing from the essential teachings of the invention. Moreover, each of the non-limiting examples described herein can stand on its own, or can be combined in various permutations or combinations with one or more of the other examples.

The above Detailed Description includes references to the accompanying drawings, which form a part of the Detailed Description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any document so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A soft tissue connector, comprising:
a substrate having first and second surfaces;
the first surface including at least one bone penetrating member for securing the substrate to a bone; and
the second surface including a topography configured to allow releasable attachment of soft tissue to the substrate.

2. The connector of claim 1, wherein the first surface includes at least two bone penetrating members.

3. The connector of claim 2, wherein a first bone penetrating member extends from a first end of the first surface, and a second bone penetrating member extends from an opposing second end of the first surface.

4. The connector of claim 3, wherein each of the first and second bone penetrating members include a bend of the substrate, the bends terminating in one or more teeth.

5. The connector of claim 1, wherein at least a portion of the substrate is flexible for fitting about a non-linear contour of the bone.

6. The connector of claim 1, wherein the substrate includes a planar sheet of material.

7. The connector of claim 1, wherein the substrate includes a pre-formed curvature.

8. The connector of claim 1, wherein the topography includes a plurality of angled, non-perpendicular projections extending from the second surface.

9. The connector of claim 1, wherein the topography includes one or more teeth.

10. The connector of claim 1, wherein the topography includes a hook or loop fastener material.

11. The connector of claim 1, wherein the topography includes a cellular structure of the second surface.

12. The connector of claim 1, wherein the substrate includes a bioresorbable material.

13. A kit, comprising:
a soft tissue connector including a substrate having opposing first and second surfaces, the first surface including at least one bone penetrating member for securing the substrate to a bone, and the second surface including a topography configured to allow releasable attachment of soft tissue to the substrate;
a fixation tool configured to secure the at least one bone penetrating member to the bone; and
a set of instructions for using one or both of the soft tissue connector or the fixation tool.

14. A method for facilitating connection of soft tissue to bone, the method comprising:
obtaining a soft tissue connector including a substrate having first and second surfaces, the first surface including at least one bone penetrating member for securing the substrate to a bone, and the second surface including a topography configured to allow attachment of soft tissue to the substrate;
securing the at least one bone penetrating member to the bone; and
attaching soft tissue to the topography, in a first position on the connector, to effectuate a first tissue tensioning.

15. The method of claim 14, further comprising testing one or more of a stability or a range of motion of a joint, associated with the bone, when the soft tissue has the first tissue tensioning.

16. The method of claim 15, further comprising, if the testing result is not satisfactory, adjusting the tension of the soft tissue by releasing the soft tissue from the topography and re-attaching the soft tissue to the topography, in a second position on the connector, to effectuate a second tissue tensioning.

17. The method of claim 16, further comprising testing one or more of the stability or the range of motion of the joint when the soft tissue has the second tissue tensioning.

18. The method of claim 14, wherein attaching the soft tissue to the topography includes releasably attaching the soft tissue to the soft tissue connector without causing tissue damage.

19. The method of claim 14, further comprising driving one or more tacks through the soft tissue and into the bone to secure a position of the soft tissue.

20. The method of claim 14, further comprising positioning a second soft tissue connector on an outward facing surface of the soft tissue and securing the second soft tissue connector to the first soft tissue connector or to the bone.
